(19) United States
(12) Patent Application Publication
(10) Pub. No.: US 2013/0225789 A1
(43) Pub. Date: Aug. 29, 2013

(54) Polyethylene Glycol Having Hetero Multiple Functional Groups

(71) Applicants: Yi SUN, Wellesley, MA (US); Zhuo Sun, Shanghai (CN)

(72) Inventors: Yi SUN, Wellesley, MA (US); Zhuo Sun, Shanghai (CN)

(21) Appl. No.: 13/408,105

(22) Filed: Feb. 29, 2012

(51) Int. Cl. C07C 271/22; C07H 21/00; C07K 17/02; C07D 495/04; C07C 247/04

(52) U.S. Cl. 530/300; 560/165; 548/304.1; 552/11; 536/23.1

(57) ABSTRACT

Novel PEG multifunctional derivatives. A PEG backbone molecule is covalently attached with at least three different functional groups, either on the same branch arm or on different branch arms.

### DESCRIPTION OF RELATED ART

The present application relates to biocompatible polymer derivatives, and more particularly to a multifunctional PEG derivative molecule that possesses three and more different functional groups on its plurality of terminuses sufficiently ready for further use.

Note that the points discussed below may reflect the hindsight gained from the disclosed inventions, and are not necessarily admitted to be prior art.

Polysaccharides and PEGs have been widely used for pharmaceutical products and cosmetical products. Their non-toxicity, non-immunogenic and water soluble features are the most appealing to the pharmaceutical and biotechnology industries. Drugs may be modified with them on the surface to increase both the life time and the efficacy inside the body. Chemical attachment of hydrophilic PEG to proteins and other molecules is of great utility in biotechnology as well, such as bio-labeling and bio-probing.

PEG polymers exist in linear forms, branched forms and/or multi-arm polyethylene glycols. The linear PEG polymers have a core structure of HO—CH₂CH₂O—(CH₂CH₂O)n-CH₂CH₂—OH. Branched PEG polymers have a core structure of R-(PEG-OH)n, R represents a core molecule, such as glycerol, pentaerythritol and n represents the number of arms, likely composed of a linear or further branched PEG structure. PEG attachment requires activation of PEG molecules with more chemically reactive functional groups. Functional chemical groups have been attached to the terminuses of PEG molecules to link or modify other molecules. For example, mono-functional polyethene glycol aldehydes are described in the U.S. Pat. No. 7,041,855 B2 to Rosen et al, in which one of the terminuses of a linear or branched PEG molecule are attached with an aldehyde group for being further used to conjugate with a therapeutic protein molecule or other molecules. Other mono-functional PEGs attached with one of the chemically reactive functional groups have been reported and commercially available. These functional groups include NH₂, COOH, Biotin, Maleimide, NHS, Fluorophors, etc.

Bifunctional PEGs with two same functional groups linked to the terminuses of a PEG molecule are described in the art, as shown in U.S. Pat. No. 5,162,430 to Rhee, et al. Moreover, PEG molecules having two different functional groups are also described in the art, for example, in U.S. Pat. No. 6,541,543 B2, a linear or branched PEG molecule is covalently attached with two different functional groups linked though a branched atom to one of the terminuses.

However, biocompatible polymers having three or more different functional groups attached on different terminuses are useful for multiple bio-probing and imaging for more accurate measurement and localization, but are not currently available.

### SUMMARY

The present application discloses a novel PEG derivative molecule that has at least three different functional groups covalently attached to the terminuses of the PEG backbone.

In one embodiment, a PEG molecule having the following structure is described:

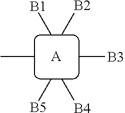

wherein A is a polyethylene glycol core, linear or multi-branched; B1 to B5 are different functional groups linked on different terminuses of A's backbone, the functional groups including —NH₂, —SH, —COOH, —N₃, —CHO, —NHNH₂, —OH, —OCH₃ (methoxyl), succinimidyl ester (—NHS), aldehyde, isocyanate, epoxy, azyde, hydrazide, maleimide, vinyl, tosyl, alkyne, vinyl sulfone, triethoxyl silane, trimethoxyl silanes, biotin, fluorophores, lipids, peptides, nucleotides, proteins, vitamin, carbohydrates, nanoparticles.

In one embodiment, a multi-branched PEG molecule A with a plurality of PEG arms has a plurality of functional groups R1 to Rx covalently attached, the functional groups being of a mixture of same group and different groups attached either to the same terminal or different terminals with a structure formula of the following:

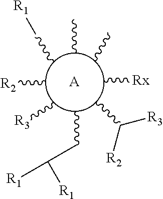

wherein A is a polyethylene glycol core, linear or multi-branched; R1 to Rx are different functional groups linked on the terminuses of a PEG arm of A's backbone, the functional groups including chemically reactive groups such as —NH₂, —SH, —COOH, —N₃, —CHO, —NHNH₂, —OH, succinimidyl ester (—NHS), aldehyde, isocyanate, epoxy, azide, hydrazide, maleimide, vinyl, tosyl, alkyne, vinyl sulfone, acrylate, triethoxyl silane, trimethoxyl silanes, etc.

In one embodiment, the functional groups also include various fluorescent dyes that has excitation/emission wavelength from UV region to near infrared region. Such dyes include fluorescein based dyes, rhodamine based dyes, cyanine dyes. There are many commercial suppliers for those fluorescent dyes such as ALEXAFLUOROA® and BODIPY® dyes from Life Technologies, Inc, Cyanine dyes from GE Life Science.

The functional groups also include bioactive small molecules such as vitamins, including biotins, vitamin D, vitamin E, and other small molecules that have specific bioactivity, such as lipids and phospholipids. The Functional groups also include peptides and oligonucleotides that have specific bioactivity. The functional groups also include carbohydrates that have specific bioactivity and metal chelates.

The disclosed innovation, in various embodiments, provides one or more of at least the following advantages. However, not all of these advantages result from every one of the innovations disclosed, and this list of advantages does not limit the various claimed inventions.
Biomolecules can be labeled with multiple different functional groups/molecules easily and readily with the described multifunctional PEG derivative reagents;
The labeling and modifying reactions can be performed with biomolecules readily in aqueous buffer without addition of organic solvents, preserving the biomolecules' biological functions.

### DETAILED DESCRIPTION OF SAMPLE EMBODIMENTS

The numerous embodiments of the present application will be described with particular reference to presently preferred embodiments (by way of example, and not of limitation). The present application describes several embodiments, and none of the statements below should be taken as limiting the claims generally.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the invention.

The terms “first,” “second,” “third,” “fourth,” and the like in the description and the claims, if any, may be used for distinguishing between similar elements and not necessarily for describing a particular sequential or chronological order. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

The terms “groups,” “functional group,” “moiety,” “active moiety,” “reactive site,” “reactive group” and “reactive moiety” are used in the art and herein to refer to distinct, definable portion or unit part of one molecule as generally used in chemistry science that confers a special and distinctive and detectable chemical or physical property to the molecule. The term “linkage” is used herein to refer to groups or bonds that normally are formed as the result of a chemical reaction and with covalent linkages, and the result is a new compound with new chemical property.

The term “biological agent” or “bioactive” is used herein to refer to any molecules that would have a biological effect if inside a living organism or would naturally interact with a biologically effective molecule in the body. Examples are proteins, peptides nucleotides, DNAs or RNAs and other polymers, vitamins, etc.

Examples are given to synthesize PEG derivatives with multiple different functional groups. It is contemplated and intended that the example reactions are given to PEG reagents, but the reactions can also be applied to polysaccharide polymers, such as dextran.

### Example 1

the synthesis of PEGs attached with three different chemically reactive functional groups, a —NH₂ group, a —COOH group and a —SH group (compound 14) is described.

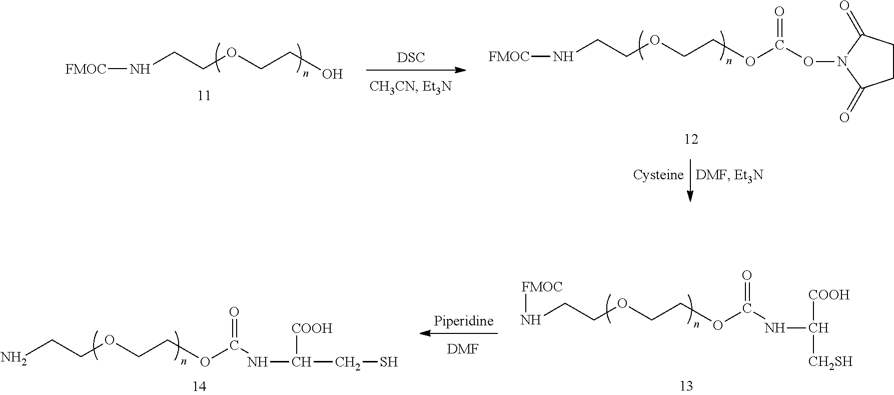

0.5 g (0.1 mmol) F_{MOC}—NH-PEG-OH (compound II), MW 5000, purchased from any chemical company, for example Merck company, was dissolved in 10 mL acetonitrile (CH₃CN) in a 50 mL glass flask. Under vigorous stirring, 0.256 g (1 mmol) disuccinimidyl carbonate (DSC) was added to the solution slowly. After addition of DSC, 0.2 mL triethyl amine (Et₃N) was added to above solution as catalyst. The reaction was allowed to proceed for 12 hours protected under argon. After the reaction, the solvent was evaporated under reduced pressure and the reaction mixture was re-dissolved in toluene, then the insoluble material was filtered and the solution was collected. Compound 12 was obtained after addition of cold diethyl ether to the solution. The process of re-dissolvation and filtration was repeated for 3 times to get rid of any impurities.

To obtain compound 13, 0.5 g compound 12 was dissolved in DMF. With vigorous stirring, 0.24 g cysteine was added to the solution, and 0.5 mL Et₃N was then added as catalyst. After 12 hour reaction, DMF was removed under reduced pressure and the reaction mixture was re-dissolved in methylene chloride and purified with chromatograph.

Compound 14 was obtained by deprotecting the Fmoc group. Compound 13 was dissolved in 10 mL DMF, and 2 mL piperidine was added to the solution, the reaction was allowed for 2 hours. The solvent was then evaporated under reduced pressure and the reaction mixture was washed with cold diethyl ether for 3 times. The final compound 14 was further purified with chromatograph and dried under vacuum. Compound 14 has three reactive functional groups: —NH₂, —SH and —COOH covalently attached to the PEG backbone.

### Example 2

synthesis of PEGs with 4 different chemically reactive functional groups is described.

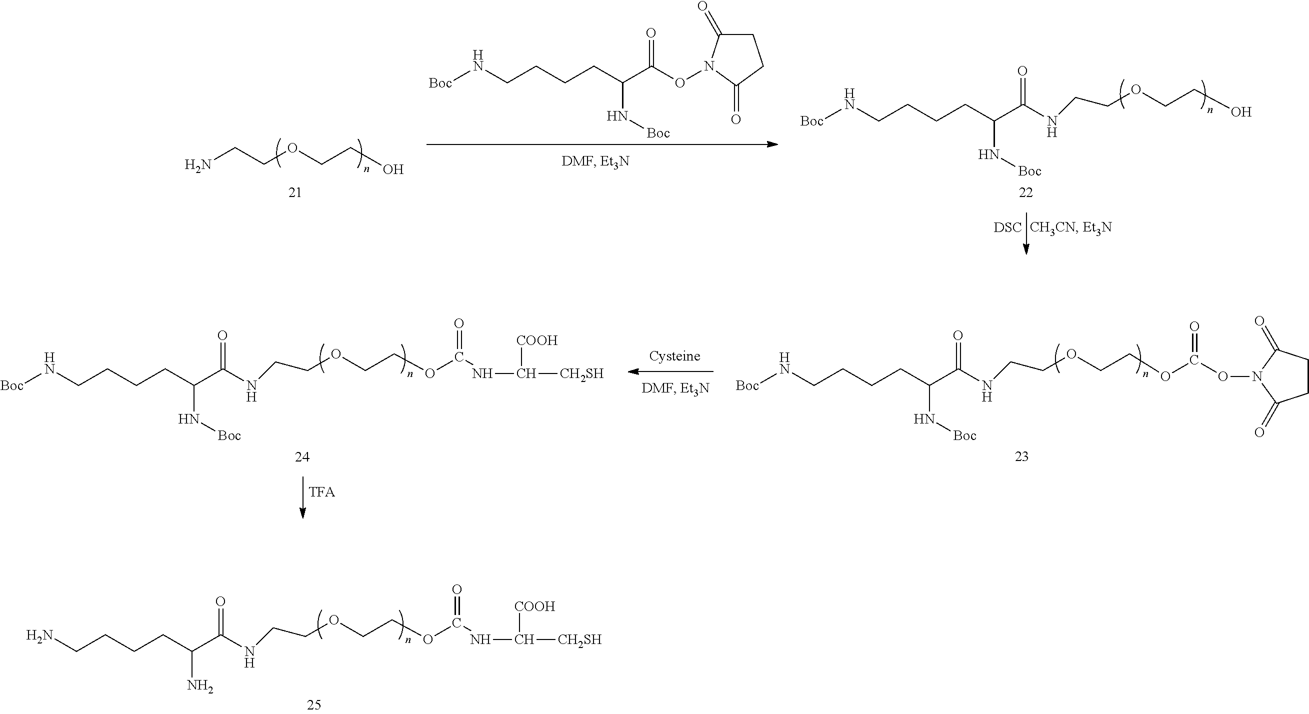

0.5 g (0.1 mmol) compound 21, a PEG molecule, MW 5000 was dissolved in 10 mL DMF. To this solution, 88 mg SIGMA-ALDRICH® Boc-Lys(Boc)-OSu was added with vigorous stirring. After all solid compounds were dissolved, 0.2 mL triethyl amine was added. The reaction was allowed to proceed for 4 hours under argon. After the reaction, the reaction solvent was evaporated under reduced pressure. The produced Compound 22 was further purified with silica chromatograph.

To prepare compound 23, 0.5 g (0.1 mmol) compound 22 was first dissolved in acetonitrile, and to this solution, 0.256 (1 mmol) DSC was added. After all solid were dissolved, 0.5 mL triethyl amine was added with vigorous stirring. The reaction was allowed to proceed for 4 hours under argon, then the solvent was evaporated under reduced pressure and the reaction mixture was purified by chromatograph. After removing solvent, compound 23 was obtained as a white solid.

Compound 24 was prepared by reacting compound 23 with cysteine in DMF, catalyzed with Et₃N. In this reaction, 0.5 g compound 23 was first dissolved in DMF, and to this solution, 0.24 g cysteine was added. After all solid were dissolved, 0.2 mL Et₃N was added as catalyst. The reaction was allowed to proceed for 4 hours under argon and compound 24 was purified by chromatograph and dried under vacuum.

Compound 25 was prepared by de-protecting Boc groups from compound 24. In this process, 0.5 g compound 24 was dissolved in 10 mL methylene chloride, then 10 mL TFA (trifluor acetic acid) was added. The resulting mixture was stirred at room temperature for 5 hours. The reaction mixture was poured to solid sodium bicarbonate that was premixed with ice, then was extracted with dichloromethane and dried with Na₂SO₄. The solvent was evaporated to produce the compound 25, which is a PEG polymer that has four functional groups, i.e. two reactive amine groups, one thiol group and one carboxylic group.

### Example 3

synthesis of PEGs with 3 non-chemically reactive functional groups is described.

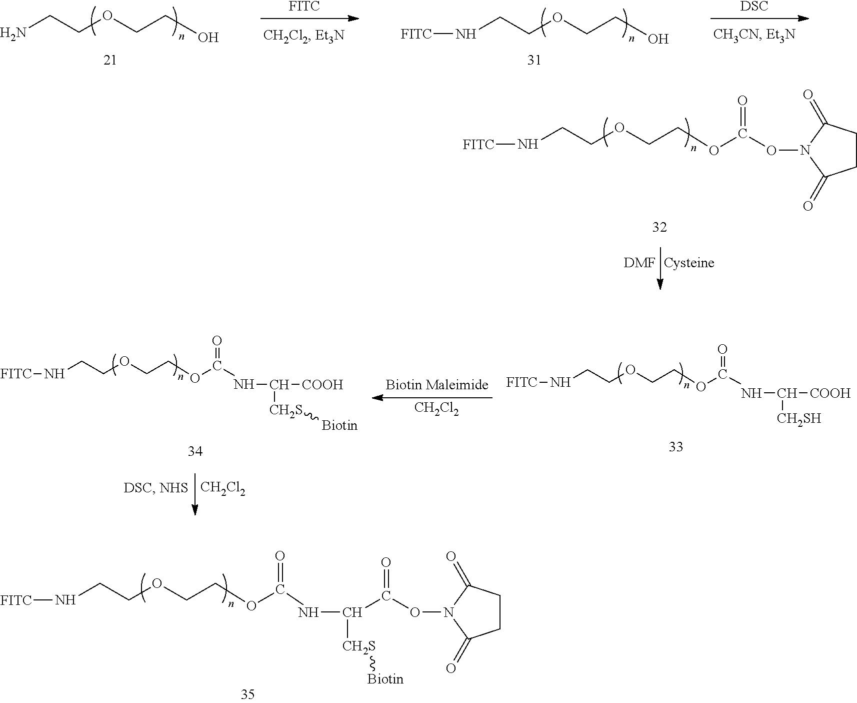

0.5 g compound 21 was dissolved in 10 mL methylene chloride in room temperature, and to this solution, 389 mg (1 mmol) fluorescein isothiocyanate (FITC, from Sigma Aldrich) was added. After all solid were dissolved, 0.5 mL Et₃N was added as catalyst. The resulted mixture was stirred in dark for 5 hours. The solvent in the reaction mixture was evaporated under reduced pressure and the solid was purified with chromatograph. After the solvent was evaporated, compound 31 was obtained as yellow solid.

Compound 32 was prepared by reacting compound 31 with DSC under the similar reaction conditions described in Example 2. Compound 33 was obtained by reacting compound 32 with cysteine in DMF under similar reaction conditions described in Example 2. Compound 34 was prepared from the reaction between compound 33 and Biotin maleimide (available from Sigma Aldrich). In this reaction, 250 mg compound 33 was dissolved in methlyne chloride, and to this solution, 200 mg Biotin maleimide was added and Et₃N was used as catalyst. The resulted reaction mixture was stirred for 12 hours in dark.

The resulted compound 34 was purified by chromatograph. Yellow solid compound 34 was obtained after solvent was evaporated. Compound 35 was prepared from compound 34 using DSC as condensing reagent. In this reaction, 100 mg (0.02 mmol) compound 34 was dissolved in 5 mL methylene chloride, to this solution, 412 mg (0.2 mmol) DSC and 230 mg NHS (0.2 mmol) was added. The resulted mixture was stirred in the dark for 24 hours. The reaction was filtered and the yellow solution was evaporated under reduced pressure. The obtained compound 35 mixture was re-dissolved in methylene chloride and purified with HPLC. Compound 35 was obtained as yellow solid after solvent was evaporated. Compound 35 has fluorescent group FITC, a bioactive biotin group, and triethyl amine protected —COOH group, ready for labeling use as described in Example 4.

### Example 4

fluorescent and biotin labeling of BSA using a PEG having hetero multifunctional groups is described. 10 mg BSA (Bovine serum albumin) was dissolved in 1 mL 10 mM NaHCO₃, pH 8.5 buffer, and to this solution, 2 mg of compound 35 was added. The resulted mixture solution was stirred for 2 hours in 4° C. After this reaction, the mixture solution was purified with Sephadex G-25 and FITC labeled BSA was eluted with PBS buffer, pH 7.4. FITC labeling rate was measured by absorption at 495 nm and biotin labeling rate was measured with streptavidin binding assay. The resulted molecule in terms of PEG backbone molecule is a PEG with a protein or polypeptide group, a fluorescent FITC group, a bio-reactive Biotin group. This molecule can be used as bio-probe to further react with other bio-molecules, for example, in this case, an antibody of BSA. If a poly-peptide, for example, a signal peptide for organelle trafficking or localization, is used in place of BSA in the above reaction, and the peptide can be recognized by another molecule, the interaction between the peptide with other biomolecules can be monitored with either the FITC group or the biotin group. If an oligonucleic acid sequence is used for labeling in the above reaction, this labeled oligonucleic acid can be used to probe its complementary sequences inside a cell or a tissue sample for bimolecular study or diagnosis.

### Example 5

the synthesis of multi-arm PEGs with 4 functional groups is described, wherein at least 2 groups are different. The following chemical reactions were conducted.

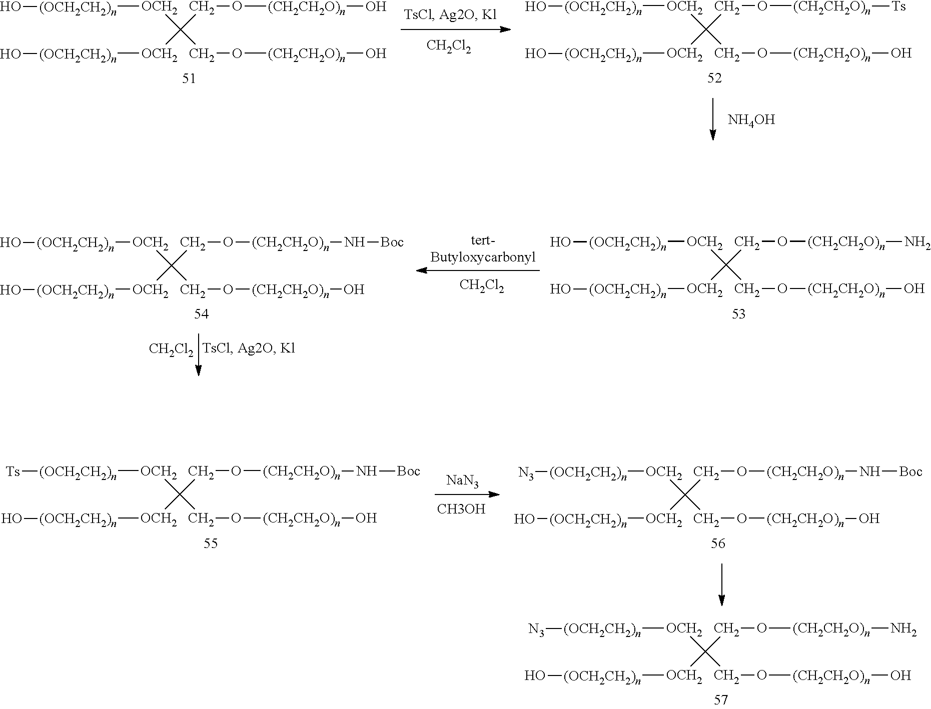

2 g (0.1 mmol) compound 51 was, MW 20000, was purchased and was dissolved in 10 mL CH₂Cl₂. To this solution, 19 mg (0.1 mmol) tosyl chloride was added slowly in ice bath, then 23.4 mg freshly prepared Ag₂O and 16.6 mg KI was added as catalyst. The resulted mixture was stirred under argon for 12 hours, was then filtered and the solvent was evaporated under reduce pressure. The obtained solid was re-dissolved in CH₂Cl₂ and purified with chromatograph. Compound 52 was obtained as white solid after solvent was evaporated.

Compound 52 was dissolved in concentrated ammonium hydroxide solution and the reaction mixture was stirred for 48 hours. The resulted reaction mixture was extracted with CH₂Cl₂ three times, dried with Na₂SO₄ and the solvent was evaporated under reduced pressure to produce Compound 53. Then the amine group in compound 53 was protected with Boc carbonyl and compound 54 was obtained. The second —OH group in compound 53 was further reacted with tosyl chloride as before and compound 55 was obtained. Azide functional group (—N₃) was then introduced to compound 55 by reacting compound 55 with NaN₃ under reflux to produce Compound 56. Compound 56 was obtained after purification. Boc group in compound 56 was de-protected with CH₂Cl₂ with 50% TFA to produce compound 57. Compound 57 has four functional groups with three different functionalities (—N₃, —NH₂, —OH), on four PEG arm branches. Compound 57 can be used to further react with fluorescent molecules, biotins, carbohydrate molecules, such as glucose, vitamins, enzyme inhibitors, cell receptor binding molecules, oligonucleotides, and etc.

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

What is claimed is:

1. A PEG derivative molecule, comprising:
a PEG polymer backbone having a first position, a second position, a third position;
a first functional group is covalently attached to said first position;
a second functional group is covalently attached to said second position; and
a third functional group is covalently attached to said third position;
wherein said first functional group, said second functional group and said third functional group are chemically different, each adding a different chemical property to said PEG polymer backbone.

2. The PEG derivative molecule of claim 1, wherein PEG backbone is a linear PEG.

3. The PEG derivative molecule of claim 1, wherein PEG backbone is a branched PEG.

4. The PEG derivative molecule of claim 1, wherein said first functional group, said second functional group and said third functional group each is chemically reactive group selected from a group consisting of —OH, —NH₂, —COOH, —CHO, —NHS, —SH, -epoxy, —N₃, alkyne, —NHNH₂, —Si(OCH₂CH₃)₃, maleimide, orthopyridyl disulfide, nitrophenyl carbonate, carbonyl Imidazole, tosylate, mesylate, acrylate, and vinylsulfone.

5. The PEG derivative molecule of claim 4, wherein said first functional group is a fluorophore with excitation and/or emission wavelength ranging from 300 nm˜1100 nm.

6. The PEG derivative molecule of claim 5, wherein said second functional group is a peptide.

7. The PEG derivative molecule of claim 5, wherein said second functional group is an oligonucleotide.

8. The PEG derivative molecule of claim 5, wherein said second functional group are metal chelate.

9. The PEG derivative molecule of claim 5, wherein said second functional group is a carbohydrate.

10. The PEG derivative molecule of claim 4, wherein said first functional group is bioactive small molecule selected from a group consisting of vitamins, enzyme inhibitors, and cell receptor binders.

11. The PEG derivative molecule of claim 1, wherein said first functional group is a fluorophore, said second functional group is a chemically reactive group selected from a group consisting of —OH, —NH₂, —COOH, —CHO, —NHS, —SH, -epoxy, —N₃, alkyne, —NHNH₂, —Si(OCH₂CH₃)₃, maleimide, orthopyridyl, disulfide, nitrophenyl carbonate, carbonyl Imidazole, tosylate, mesylate, acrylate, and vinylsulfone, and said third functional group is a biotin.

12. The PEG derivative molecule of claim 1, further comprising:
a fourth functional group being covalently attached to a fourth position on said PEG polymer backbone;
wherein said fourth functional group is chemically different to said first, said second and said third functional group, adding a fourth chemical property to said PEG polymer backbone.

13. The PEG derivative molecule of claim 12, wherein said fourth functional group is selected from a group consisting of —OH, —NH₂, —COOH, —CHO, —NHS, —SH, -epoxy, —N₃, alkyne, —NHNH₂, —Si(OCH₂CH₃)₃, saleimide, orthopyridyl disulfide, nitrophenyl carbonate, carbonyl imidazole, tosylate, mesylate, acrylate, and vinylsulfone.

14. The PEG derivative molecule of claim 12, wherein said fourth functional group is a fluorophore with excitation/emission wavelength from 300 nm˜1100 nm.

15. The PEG derivative molecule of claim 12, wherein said fourth functional group is a peptide.

16. The PEG derivative molecule of claim 12, wherein said fourth functional group is an oligonucleotide.

17. The PEG derivative molecule of claim 12, wherein said fourth functional group are metal chelate.

18. The PEG derivative molecule of claim 12, wherein said fourth functional group is a carbohydrate.

19. The PEG derivative molecule of claim 1, wherein said first functional group a fluorophore, said second functional group is a chemically reactive group, and said third functional group is a peptide.

20. The PEG derivative molecule of claim 1, wherein said first functional group a fluorophore, said second functional group is a chemically reactive group, and said third functional group is an oligonucleotide.

21. The PEG derivative molecule of claim 1, wherein said first functional group a fluorophore, said second functional group is a chemically reactive group, and said third functional group is a carbohydrate molecule.
